(19) United States
(12) Patent Application Publication
(10) Pub. No.: US 2014/0243261 A1
(43) Pub. Date: Aug. 28, 2014

(54) Method for treating diabetes and fatty liver disese by stimulating cellular metabolism

(71) Applicant: G. Ford Gilbert, McClellan, CA (US)

(72) Inventor: G. Ford Gilbert, McClellan, CA (US)

(21) Appl. No.: 13/999,453

(22) Filed: Feb. 26, 2014

Related U.S. Application Data
(60) Provisional application No. 61/758,735, filed on Feb. 26, 2013.

(51) Int. Cl. A61K 38/28; A61K 31/7004; A61K 38/22; A61K 31/70

(52) U.S. Cl. CPC A61K 38/28; A61K 31/70; A61K 31/7004; A61K 38/22
USPC 514/6.5; 514/23; 514/9.7

(57) ABSTRACT

The present invention is a method of treating various diseases and conditions through administering a substantial caloric load coupled with administration of hormone to stimulate the body to convert that caloric load and increase metabolism in at least some cells. Body functioning converts intracellular adenosine triphosphate (ATP) as a part of normal cell function. Complex control systems balance caloric inputs by managing internal hormone levels to consume, store, or eliminate various types of caloric inputs. This invention stimulates these systems to increase metabolism of cells within the body and thereby treat various diseases and conditions. Many patients find their metabolism after treatment to be in better balance, and the patient's health improved.

### CROSS-REFERENCE TO RELATED APPLICATIONS

This application is a continuation of provisional application Ser. No. 61/758,735, filed Feb. 26, 2013, and a continuation in part of application Ser. No. 13/341,813, filed Dec. 30, 2011, which applications are incorporated herein by reference in their entirety and for all purposes.

### FIELD OF INVENTION

This invention relates to medical treatments, and more specifically to a method of treating various diseases and conditions through administering a substantial caloric load coupled with administration of hormone to stimulate the body to convert that caloric load and increase metabolism in at least some cells. Body functioning converts intracellular adenosine triphosphate (ATP) as a part of normal cell function. Complex control systems balance caloric inputs by managing internal hormone levels to consume, store, or eliminate various types of caloric inputs. This invention stimulates these systems to increase metabolism of cells within the body and thereby treat various diseases and conditions. Many patients find their metabolism after treatment to be in better balance, and the patient's health improved.

This invention relates more specifically to a medical infusion system which provides infusions controlled in timing and amounts to develop whole blood oscillations of free hormones, including insulin. These infusions are delivered to rise in amounts to support glucose control when the patient ingests a substantial carbohydrate load. This load can be in an amount equal to a significant percent of the recommended daily allowance of carbohydrates for the person, considering the weight of the patient. The infusion delivers levels of hormones to stimulate oscillations in blood glucose levels over a significant percent of normal blood levels. This system and method achieves improved and elevated intracellular adenosine triphosphate levels which improve metabolic activity, leading to effective treatment for various diseases and conditions.

### BACKGROUND OF THE INVENTION

The historical means of delivering medicines have been arbitrarily divided into two types of delivery. The first is pharmaceutical delivery of compounds through chemically based systems using various carriers with specific chemical properties to control the uptake of the active chemicals. The compounds and carriers react in predictable responses to the tissues which are proximate and affected by the active compound, thus providing the modulated delivery of medicines.

The second means of delivering medicines uses various mechanical, absorptive and electro-mechanical systems to modulate the delivery, which differ from chemical delivery systems. The current invention in one preferred implementation provides a bolus delivery system which delivers selected amounts of one or more hormones, and particularly insulin, selected and administered to promote high blood glucose levels, followed by low blood glucose levels, repeatedly cycling high and low blood glucose levels over a cycle time frame less than hours, and in a preferred embodiment on the order of less than about 20 minutes, and more preferably about 4 to about 8 minutes. This is intended to achieve elevated adenosine triphosphate (ATP) levels within cells receptive to the hormone.

Some medical devices have attempted to adjust the delivery of hormones including insulin in conjunction with determining blood glucose levels by various means. One example infuses insulin by Continuous Subcutaneous Insulin Infusion (CSII) such as the Medtronic wearable insulin pump with glucose reading information, as in Starkweather, U.S. Pat. No. 6,694,191. There are other truly “closed loop” systems such as the Miles Biostator device as in Clemens, U.S. Pat. No. 4,055,175. While the approach of these devices may constitute an improvement over devices which have no bio-feedback of the patent's response, they are not designed to and do not result in body-wide oscillations of hormones which induce improved adenosine triphosphate levels as does the present invention.

Prior delivery systems including the above closed loop systems ignore the reality that the nominal homeostasis of hormones actually has ever-changing secretion rates of hormones associated with the serum levels of ever-changing glucose levels of the patient.

It might be assumed that increasing the ability to produce adenosine triphosphate would be counter indicated for some abnormal medical conditions but such is not the case. The patient's then-existing physical condition constantly changes on an hourly or even minute-by-minute basis. Improved adenosine triphosphate production is consistently beneficial in all physical conditions yet measured.

Other delivery systems use standardized, averaged bioavailability uptake information on a broad range of patients to determine the recommended dosing of medicines and hormones based chiefly on the patient's weight, and then adjust the treatment amounts, if at all, depending upon the patient outcomes after administration. However these systems do not attempt to achieve gradient changes found in hormone homeostasis, which is actually dynamic. These systems are unable to respond to the increase or decrease of available glucose and receptor activities that can change in mere minutes, and thus do not result in increased adenosine triphosphate production by the mitochondria of the cells.

Examples of physiological rhythms include almost all endocrine functions from the more obvious circadian rhythms including varying insulin secretion rates, to the very subtle rhythms related to hormonal uptake activities. Other current hormone administering systems ignore these rhythms, and in fact most seek to establish and maintain a fixed level within relatively small variation.

In the field of pumping, there have long existed numerous ways to pump and infuse liquids for medical use, beginning with a simple syringe or pipette and progressing to highly sophisticated electromechanical software correcting systems of gates and valves with many moving parts and checking systems. In addition there exists a large number of blood and tissue testing diagnostic technologies which can detect, identify and quantify the presence and levels of various substrates in plasma and whole blood. The present inventor's pump described in Zaias et al, U.S. Pat. No. 6,565,535, is particularly useful.

In the past, improvements in basic pumping systems have centered on the use of electronic controls to compensate for the mechanical limitations of pumping systems which generally use non-rigid materials that introduce variations in delivery. Many of these more sophisticated pumping mechanisms have valves and chambers which disturb the reagents normally used in such devices. Such designs sometimes seem to be for the sole purpose of having a proprietary design. These are not preferred systems for the infusions to result in discrete oscillations.

The approach to achieving accuracy in pumping has historically been to slow the delivery so that a more precise metering could take place. This is not preferable in this invention. Generally available products only offer pump accuracy specifications of plus-or-minus 2 to 5 percent, over the entire reservoir, not for each bolus. Individual deliveries at moments in time thus are much less uniform and accurate. The preferred embodiment has achieved both accuracy and the required immediate infusion keeping the bolus together during the infusion.

Prior systems of various types have produced varied and uncertain levels of temporary physiological changes. For example the Biostator was the first device which achieved constant euglycemia which was, at that time, the only goal and desired state. However, consistent euglycemia which is achieved by a constant match of insulin to glucose is not found in healthy man, does not avoid or remedy the various complications of metabolic diseases, and does not provide increased adenosine triphosphate production. It does not achieve measurable true hormone and fuel homeostasis which is very dynamic and ever changing.

This is also true of the Medtronic approach mentioned above, which also does not provide the oscillations available through using this invention to normalize metabolism by providing nominal or increased levels of adenosine triphosphate, but rather achieves a constant euglycemia without the cessation of complications, and with the “poor outcomes” associated with diabetes and other metabolic diseases.

Hormones are proteins. Many are relatively easily damaged with many types of gate, valve or force which causes shearing upon the opening and closing of the mechanism used to stop the flow. These proteins have the ability to aggregate and become less effective, thereby giving to the patient a treatment which has changed in its effective concentrations. Shear and aggregation can also occur with flows through narrow high-pressure regions. The current invention avoids these problems of shear, and degrading pressures by having no gate or valve, and by having no pressurized closing areas in the travel of the fluid.

Some hormones are delivered in a relatively inaccurate concentration, due to the forces of ionization and collection of medicines on the surfaces of the bag or container being used as a reservoir to store and deliver the medicine. The medicine can collect on the sides of the container, and be delivered only in a relatively unknown and possibly short period of time. The current invention avoids this problem by allowing a very accurate and controlled delivery and by avoiding a high level of dilution.

Many pumping devices which use syringes have no ability to overcome the natural slip-stick or chatter associated with the storage of energy in the elastic and pliable surfaces and structures, allowing for the syringe moving face (“Plunger”) to move forward in irregular motions. Hysteresis and the natural tendency of Plungers not to move until a force overcomes the inertia and sticking forces cause deliveries by most syringe pumps to be sporadically subject to differing levels of sticking (sticktion). And yet, because of the need for accuracy the other types of infusion devices are not as suitable. When these other types of infusion devices overcome this inertia and hysteresis, they tend to overrun and deliver at different speeds.

### SUMMARY OF THE INVENTION

Providing precise bolus deliveries of hormones intravenously, including insulin boluses, can achieve therapeutic rhythms (oscillations). Adjusting the levels and durations between boluses can be selected to correspond to a physiologically normal response to plasma glucose increases found after ingestion of a high carbohydrate meal, One useful meal size is on the order of about forty percent (40%) of the total suggested daily calories for the patient, considering weight and as desired other factors which a nutritionist or staff person might find useful. Such a delivery addresses and uses individual physiological responses in the patient. Meal and bolus patterns are tailored to encourage the blood glucose to remain within a range of approximately 60 to approximately 300 mg/dl. Wider ranges can be and have been used. This has been tested up to about 400 mg/dl and could be higher still. This induces increased intracellular adenosine triphosphate (ATP) conversion from carbohydrates. This supports previously unattainable outcomes through the improved metabolic integrity of the cell.

In the present invention, providing oscillation and adjustment of patterns of treatment to cover a pre-established carbohydrate load is new. The oscillations are changed through the infusion level and timing of the delivery, and are adjusted to cover a normal calorie carbohydrate load. This is directed to improve therapeutic responses. The present invention provides a means to deliver this system of timed and accurate hormone boluses resulting in responsive oscillations within the whole blood to treat a number of different conditions and disease states by providing changes that are somewhat like changing secretion rates found in healthy humans.

Boluses of hormone are administered in a preferred embodiment with sufficient flow and pressure to maintain their relative concentration intravenously when first entering the vein. Boluses are tailored to provide oscillations (rhythms) which enhance the metabolic integrity of cells and organs through increased levels of adenosine triphosphate. The purpose of this invention is to provide medical devices and methods which achieve these therapeutic benefits, and particularly new control regimes for selective infusion profiles.

Ongoing determination of the levels of carbohydrates in whole blood allows the amount and duration of hormone infusion to match the ingestion of foods or fluids containing carbohydrates and other substrates. In the preferred embodiment, infusing the hormone intravenously makes that determination of carbohydrates more easily achieved by using the existing intravenous access to sample blood to reverse the infusion system to take a sample and make that determination.

### DESCRIPTION OF THE DRAWINGS

A preferred embodiment of the present invention is further described in connection with the accompanying drawings, in which:

FIG. 1 is a perspective view of a preferred embodiment of the medical infusion and aspiration system.

FIG. 2 is a perspective view of a preferred embodiment of the cartridge.

FIG. 3 is a perspective view of a preferred embodiment of the housing and plunger.

FIG. 4 is a perspective view of another preferred embodiment of the medical infusion and aspiration device.

FIG. 5 is a perspective view of a third preferred embodiment of the medical infusion and aspiration device.

FIG. 6 is a perspective view of a fourth preferred embodiment of the medical infusion and aspiration device.

FIG. 7 is a perspective view of a fifth preferred embodiment of the medical infusion and aspiration device.

FIG. 8 is a perspective view of an embodiment of the medical infusion and aspiration system having two cassettes being driven independently.

FIG. 9 is a perspective view of an embodiment of the medical infusion and aspiration system having two cassettes coupled by mechanical linkage.

FIG. 10 is a graph of illustrative infusion profiles and free levels of hormones when starting with a glucose load. Several profiles are shown in FIGS. 10A, 10B, 10C and 10D.

### DETAILED DESCRIPTION OF THE INVENTION

The present invention is directed to a method of treating metabolic diseases. In particular, the present invention is helpful in the treatment of diabetes. In another embodiment, the invention is useful in the treatment of fatty liver disease.

In understanding preferred implementations, the following background can be useful.

Blood levels of glucose are sometimes expressed as mg/dl (milligrams per deciliter) and sometimes as mmol/L (millimoles per liter). Table 1 lists some useful ranges in columns 1 and 2, labeled in these units, and showing the corresponding values in these respective units.

In considering possible treatment options, it has been observed that a useful amount of carbohydrate to administer varies with the observed blood glucose level. Referring to Table 1, if a patient has a blood glucose level of 145 mg/dl (which is in the typical normal range), the initial treatment suggestion is to administer 50 grams of carbohydrates for a treatment session. If blood glucose levels are higher, for example 325 mg/dl, then less carbohydrate is given—20 g per table 1. The column of calories is a simple calculation of the number of calories in the corresponding value of grams of carbohydrate. For example, 20 grams of carbohydrate is equivalent to 76 calories.

TABLE 1

| | | Grams | |
|---|---|---|---|
| mg/dl | mmol/L | Carbohydrates | Calories |
| below 100 | Below 5.5 | 60 | 220 |
| 100-149 | 5.5-8.2 | 50 | 190 |
| 150-199 | 8.3-11 | 40 | 152 |
| 200-249 | 11.1-13.8 | 30 | 114 |
| 250-299 | 13.9-16.6 | 25 | 95 |
| 300-349 | 16.7-19.3 | 20 | 76 |
| 350-399 | 19.4-22.1 | 15 | 57 |
| 400-550 | 22.2-30.5 | 10 | 38 |

A basic treatment sequence, in one preferred embodiment, is as follows.
Settle the patient and establish an intravenous (IV) flow.
Measure blood glucose.
From Table 1, arrange for the specified grams of glucose be made available to the patient.
Have the patient consume the glucose relatively quickly (time scale of minutes). Drinking an appropriate amount of GLUCOLA at a convenient rate, such as over about 30 to 90 seconds, is a useful range and route of administration.
Start IV boluses of hormone. Insulin is a useful and typical hormone for this application.
Administer hormone at the selected quantity, rate, and pattern. These details are discussed in more detail in the following sections. A typical sequence will administer the selected amount of insulin (milliunits, determined as per kilogram body weight of the patient). A useful bolus is fairly quick, such as less than five seconds. Bolus injections are repeated periodically. A useful scheme is to give boluses every six minutes, over the course of one hour. It can be useful to give boluses more rapidly. Every four minutes is a useful number. Spacing of approximately three to approximately ten minutes can be useful, and we have not tested the outer ranges of usefulness. Spacing of about every four to six minutes has been observed to be particularly useful.

Administering a bolus of insulin will typically cause a physiological response triggering the body to consume glucose from the blood. One primary mode for this glucose consumption is for various cells and tissues to increase metabolic activity. This is a major goal of the present invention.

A reason to give the carbohydrate supply is to balance the insulin administration. Carbohydrate consumed by drinking becomes available in the blood fairly quickly, raising blood glucose levels. Administering an insulin pulse decreases blood glucose levels as cells increase metabolism.

Pulses are given repeatedly over a period of time. One hour is a useful time. At every six minutes, this is 10 pulses (the last at 54 minutes after the initial pulse).

A single such cycle is useful in its own right, such as over about one hour.

It is preferable to continue with a rest cycle, giving no insulin. It is useful to have the patient consume additional glucose at the beginning of this rest cycle. We have found giving a total of about 100 g glucose over a combined pulse and rest cycle gives beneficial effects. Thus if the initial measurement of blood glucose level was 325 mg/dl and from table 1 we administered 25 g glucose with the pulses, in the subsequent rest period we would give an additional 75 grams of glucose, for a total of 100 grams for that pulse/rest cycle.

The rest cycle can be of any practical time. We have found 10-60 minutes to be conducive to promoting cellular response, as evidenced by tissue and blood response. We have not tested or challenged any limits to this rest duration. The rest cycle does not need to be the same between multiple cycles on a given day.

For treating Type 1 diabetes, we find it useful to repeat this pulse/rest cycle three times. After the first rest cycle, blood glucose is measured again, a carbohydrate load is selected, and another series of insulin pulses is administered. In the second rest cycle, additional glucose is consumed, as just described.

In most instances, a third pulse/rest cycle is initiated shortly after the second rest cycle. Such a second rest cycle can be for a moderately short time, such as 10 minutes. For some patients, it may be useful to conduct an additional fourth pulse/rest cycle.

At some point, body processes saturate and glucose ceases to be consumed by the cells and starts to be stored as fat. The number of cycles chosen is selected in consideration of total glucose loading, blood levels, and the experience of the operator. Three cycles seems to work well for most patients.

A primary purpose of this carbohydrate loading and hormone pulsing is to induce oscillations in blood glucose levels. This has been shown to have a beneficial effect on cells and tissues.

Oscillations are induced by starting and stopping infusion as described herein, such as approximately every 4 to 8 minutes, normally every 6 minutes. The duration is a momentary (brief) infusion of for example less than 5 seconds of continuous infusion followed by no infusion for that pulse event time (e.g. 4 to 8 minutes). The volume of insulin is that needed to deliver the desired amount of insulin. That amount per pulse preferably is the range of about 10 to 60 or even about 70 milliunits insulin/kg. We have not established an absolute upper or lower limit for the milliunits of insulin, infusion time or rate, pulse spacing, or number of pulses within a pulse cycle, infusion or rest periods, or varying parameters between cycles.

One useful way of assessing metabolic activity and health is to measure the volume of Carbon Dioxide Production and a Resting Energy Ratio (REE). In example (a) below, CO2 production initially equals 0.124 L per 30 seconds of exhaled breath, with a Resting Energy Ratio of 0.66. The ratio is how one can test the amount of carbohydrate metabolized and thus producing ATP to verify the patient is metabolizing carbohydrates. Below 7.0 is lipid metabolism, and above 9.0 is carbohydrate metabolism.

From Wikipedia:

The permanent gases in gas we exhale are 4% to 5% by volume more carbon dioxide and 4% to 5% by volume less oxygen than was inhaled. This expired air typically composed of:
78% nitrogen
13.6%-16% Oxygen
4%-5.3% Carbon dioxide
1% Argon and other gases

It is useful for the metabolic assessment of the present invention to express exhaled CO2 in volume/time, such as L/30 sec. This assessment varies by patient, according to their size, lung capacity and other factors understood by one skilled in the art of pulmonary function and metabolism.

A useful tool for assessing this is from Vacuumed. The Vacuumed website (www) vacuumed (corn) includes a good discussion of the resting metabolism measurement.
VO2=oxygen uptake (ml/min)
VCO2=carbon dioxide output (ml/min)
Respiratory quotient (RQ)=VCO2/VO2

From Vacuumed literature:

Interpreting the measured REE includes comparing the results to the predicted level of energy needs for that individual. Determining the 24 hour calorie intake of that individual from either an oral diet or specialized nutrition therapies (through feeding tubes into the gastrointestinal tract or intravenous administration) is required. It is important to assess the RQ to make certain it is within physiological range and consistent with the person's calorie intake and medical history. The physiological range of RQ is 0.67 to 1.3. This value represents the combination of carbohydrate, fat and protein being used for energy. If the RQ is greater than 1.0, decrease the total calorie intake and adjust the carbohydrate to fat ratio. If the RQ is less than 0.81 increase the total calorie intake, dependent on the goal for the nutrition therapy. Food sources and conditions have specific RQ values that are useful when interpreting the REE and making recommendations for changing dietary goals and feeding regimens. (1, 4, 5) Energy source/condition RQ prolonged ketosis <0.70 fat 0.70 underfeeding <0.71 protein 0.80 mixed energy 0.85 carbohydrate 1.00 fat storage >1.00

Vacuumed also cites the following paper.

“Resting energy expenditure-fat-free mass relationship: new insights provided by body composition modeling.” Zimian Wang, Stanley Heshka, Dympna Gallagher, Carol N. Boozer, Donald P. Kotler and Steven B. Heymsfield Am J Physiol Endocrinol Metab 279:E539-E545, 2000.;

The following describes some useful information about preferred implementations and embodiments of the present invention.

### 1. The Treatment Form

a. The treatment form has 3 one-hour sessions with from 10 to 60 minutes rest between treatments.
b. TREATMENT GOAL: The goal of the treatment is to obtain increased ATP production from carbohydrates, by metabolizing approximately 100 g of glucose for each hour including the rest period. A desirable treatment blood glucose for oral carbohydrates is 150 mg/dl or 8.3 mmol/L of blood glucose. That equates to approximately 380 calories of carbohydrates for each 1-hour treatment session, or a total of 1,140 calories total for the 4.5 to 5.5 hours.
c. In one embodiment, patients' serum glucose level is monitored every thirty minutes to determine carbohydrate uptake and utilization. Resting metabolic measurement by an indirect calorimeter such as the Vacuumed Metabolic Measurement system provides the level of production of ATP from carbohydrates to validate the increase.
d. In order to maintain serum glucose levels sufficient to provide for a carbohydrate load for the production of ATP, the serum glucose is maintained at 150 mg/dl or 8.3 mmol/L or more by starting at 10 milliunits per kilo and increasing by 2 milliunits each 30 minutes until the desired level is achieved. This level is constantly adjusted each 30 minutes until the nominal load is obtained, and then continues for the remainder of the treatment, and is used for subsequent treatments. Once a treatment level is obtained, that level historically remains relatively constant and only drifts up or down by 20% over a period of months, unless the patient is infected with a virus or other temporary metabolic interfering condition, i.e., the levels once obtained historically do not change significantly, notwithstanding the fact that serum glucose typically varies from 3.3 mmol/L (60 mg/dl) to 22.2 mmol/L (400 mg/dl) without patient distress, One of the outcomes of this treatment is for the patient to achieve relatively normalized serum glucose levels within that of a normal person of 4.5 to 6.6 mmol/L (80 to 120 mg/dl) with conventional management post treatment.

For treatment example if the serum glucose is 11.1 mmol/L (200 mg/dl) the infusion would be increased by 2 milliunits/kg, 30 grams of carbohydrates administered by mouth, and a subsequent measurement of blood glucose taken 30 minutes later.

If the serum glucose was near hypoglycemia levels, i.e. 3.3 mmol/L (60 mg/dl) then 60 grams of carbohydrates would be given by mouth and the insulin reduced by 2 milliunits/kg or more if needed to avoid hypoglycemia symptoms.

In all cases, because intravenous infusions are processed by the body within 6 to 20 minutes a response is seen by the next 30-minute serum glucose measurement.
Carbohydrates of 10 to 60 grams are administered each 30 minutes, determined by the serum glucose so that the patient remains in the treatment level of 8.3 to 11 mmol/L (150 to 200 mg/dl). This is administered by staring with a the formula below and adjusting in 10 grams changes as needed to achieve the treatment range. This administration is flexible and can be modified to provide a greater or lesser carbohydrate load in order to achieve the desired changes in ATP production.

### 2. Adjusting Glucose and Insulin:

a. The Bionica Pump uses the weight of the patient to calculate a range of doses so that a dose from 10 to 60 milliunits/kg body weight is possible. This is why the patient's weight is entered into the pump and the pump suggests a concentration.
b. The dose for each patient starts at the minimum 10 milliunits/kg and is gradually brought up to that level of insulin which keeps the blood glucose of approximately 8.3 to 11 mmol/L (150-200 mg/dl), and still has the patient take in app 100 g of glucose for each one hour treatment and rest period.
c. The best way to think of adjusting the dose of insulin is to imagine one cube of the body of the patient which equals 1 kilo. (a patient of 75 kg has 75 of these cubes, a patient of 120 kilos has 120 of these cubes). The dose is how much insulin each cube is going to receive, (no matter how many cubes that patient has).
d. Blood glucose is taken every 30 min., and that is when oral glucose is given per chart provided above Table 1, and changes of insulin are made as needed.
e. Increases and decreases of insulin should be in increments of 2 milliunits/kg, to 4 milliunits/kg. Most changes should be in 2 milliunits/kg and the patient should have a high blood glucose (13-19 mmol/L) before a change of 4 milliunits/kg is made.
f. It is necessary to balance the amount of sugar and the amount of insulin with the blood glucose in the ideal treatment range of 8-11 mmol/L and still have the patient take in 100 g of glucose each treatment and rest period.
g. After the first day of treatment, a level of insulin dose will have been achieved, and the patient should start the next day's treatment at that rate.
h. During the first treatment, of each day, because of the lag of insulin acting in the body, the effect of the insulin will not be felt for at least the first 30 minutes. Thus, the insulin amount should not be adjusted until the end of the first hour of treatment because of that lag. Rather, the amount of glucose should be adjusted to keep the blood sugar above 8.3 mmol/L (150 mg/dl)
i. If over 120 grams of glucose is given during a treatment/rest period, then the amount of insulin should be reduced by 2 milliunits/kg intervals. This can be adjusted every 30 min. as needed.
   3. Unusual Dosing during Second Treatment Day.
a. Patients preferably are treated two days in a row in order to trigger what may be a somewhat dormant glycogen storage and carbohydrate utilization cycle.
b. The second day that patients are treated, patients may sometimes have difficulty in processing 100 g of glucose because their muscles and liver have already been packed as much as possible with stored glucose from the previous treatment.
c. Thus, the amount of insulin being given may go higher during this second of two days, and it may not be possible to use a total of 300 g of glucose. This is not a serious concern. The patient is almost fully stored with glycogen, something they may have not been able to do for some time, even many years.
d. Even if they may not be able to process much carbohydrates, their RER will show that they are able to burn sugar very well (usually raising into the 0.9 to 1.0 range).
   4. Ongoing Treatments (After the first 2 days of Treatment):
a. The amount of insulin that it takes for patient to process approximately 100 g of glucose during each of the 3 treatment/rest periods typically will not vary greatly from week to week. We expect patients who are very insulin resistant to see in early treatment the amount of insulin dose go down over several weeks, but are likely to always need more insulin than patients who were not insulin resistant.
b. The practitioner should watch for trends of needing less and less insulin, just as they watch for trends of needing less and less medication (particularly antihypertensive medications).
c. Treatments at intervals of once every one to two weeks on average are preferred. As treatments have proceeded, after one to two weeks of no treatment, the initial insulin dose per kilo often is preferably the amount of insulin used during the prior week. For example, if a patient was given 28 then 26 then 24 milliunits/kg the prior week's treatment cycles (typically 3 cycles per day), it would be logical to start a next cycle series with a dose of 28 and adjust as needed. However, if the prior week showed that it took over 350 grams of carbohydrates to keep that patient above 8.3 mmol/L, (150 mg/dl) then a logical starting treatment dose would be 26 or 24 milliunits/kg.
d. It is helpful to build a profile from week to week (or session to session), and see how much insulin it takes to have the patient use 260 to 350 grams of sugar.

### 5. Type 1 Diabetes:

a. Type 1 diabetics can have more difficulty maintaining a balance blood glucose. This is because patients with type 1 diabetes do not store glycogen in their liver. This should improves after they have been on the treatment for several weeks to several months.
b. Type 1 diabetics will often drop blood sugar more quickly than patients with type 2 diabetes.
c. Start a type 1 diabetic patient more slowly and reduce insulin more quickly than 2 milliunits/kg at the sign of hypoglycemia.
d. It is recommended to check the blood sugar of type 1 diabetic patients if they seem to be different, sweat, complain that they are dizzy, or have any of the other symptoms of hypoglycemia.
e. Type 1 diabetics should check their blood sugar every 30 minutes after leaving the clinic until their blood glucose is in normal range.
   6. Unusual Occurrences: (These are some of the things that can happen)
a. Nausea. If the patient becomes nauseous, then the treatment can be stopped while in progress by merely entering Stop and Enter on the hormone bolus pump. When the patient feels better, the pump can be restarted.
b. Diarrhea. Some patients have not had any carbohydrates for long enough that diarrhea becomes an issue during the second or third treatment/rest period. Patients should be assured that diarrhea should go away. In general, and unless otherwise indicated, they can also be given in the diarrhea medicines.
c. Infiltration. The IV site should be reviewed any time that the amount of insulin given to a patient would normally bring down their blood sugars, but the blood sugars are remaining high or going higher still.
d. Pump Batteries. Bionica suggests that users keep a charged battery in each pump between uses. This is because there is an internal lithium ion battery that keeps the programming for each patient if the 9 V battery needs to be replaced. If a 9 V battery is not kept in the pump between uses, the lithium ion internal battery will be used and eventually needs to be replaced. When the internal battery is depleted, the warning “bbb” will appear and the pump will not work. At this point, the pump must be returned to Bionica for a new internal battery.
e. Early stopping of a treatment within a pulse cycle. Sometimes, a patient will need to leave or have their treatment interrupted. If this happens, there will be a number of pulses that were not given, and the internal battery will remember these undelivered pulses and tried to deliver them the next time the pump is used. Any time a treatment is interrupted, the syringe should be removed and the pump turned back on to finish delivering the undelivered pulses (after one hour at most, the full treatment regimen will have been given and the pump can be used again as if the treatment had never been interrupted.)

Discussion:

Some specific examples are included below, after further description of a useful apparatus.

The present invention is a means of inducing increased adenosine triphosphate production by the mitochondria within cells by introducing a selected amount of high carbohydrate foods approximating normal requirements for the treatment period and by providing boluses of hormones and covering oscillations matching the whole blood oscillations. This treatment period is about approximately 5 to 6 hours in one preferred implementation. Ongoing monitoring biological information on circulating glucose is maintained and the hormone infusion is adjusted to keep the glucose within a desired range. The rhythmic bolus delivery provides momentary changes much like a normal secretion rate, which gradient induces increased adenosine triphosphate levels, a physiological response that is not available with conventional, subcutaneous and thus more gradual, sustained or unchanging rates of hormone delivery.

These momentary changes are useful to this invention. This adjusts the hormone delivery with sufficient pressure for the bolus to remain intact. Delivery is adjusted in both duration between boluses, and in the amount of hormone contained in the bolus, causing differently spaced and differently peaked oscillations of free levels of the hormone causing a dynamic type of hormone and glucose homeostasis, and achieving superior levels of adenosine triphosphate production within the cells of the patient.

“Dynamic” and “homeostasis” are broad terms. Earlier publications show that strict or narrow homeostasis in glucose levels is generally not helpful in promoting general health. In a normal human, glucose levels do vary between eating and fasting periods. The present invention induces such dynamic changes, averaging to a level that is physiologically compatible and typical for what we would expect for the patient in a healthy state.

These oscillations and resulting adenosine triphosphate levels induce normal responses by the cells and cause general corpus-wide benefits including therapeutic effects. Disease states or metabolic deficiencies often are quickly improved or ameliorated. Tissue responses to improved adenosine triphosphate are inherently remedial. It is the change in the value of available “free” hormones, not the total amount of free hormones in the patient's system which help the body to become dynamically homeostatic and thus nominal.

The cells, with increased adenosine triphosphate are more able to resist disease, heal themselves, and avoid accelerated apoptosis. This is part of normal cellular activities as the role of DNA is to provide a directive to the cell of its nominal functions. When a cell is provided nominal or additional adenosine triphosphate through its own mitochondria, the activities of that cell are improved.

One example of the present invention is the treatment of metabolic diseases, metabolic syndromes, nervous system dysfunctions, burn victims and healing disorders. Individually tailored and adjusted delivery, to cause oscillations of circulating hormone levels such as insulin, results in the expression, repression, activation and inactivation of enzymes and other physiological reactions to achieve improved adenosine triphosphate production within the cells, and thus a result of greater cellular energy and heath. Some of the disease states which the inventor has determined respond to improved adenosine triphosphate levels are heart and cardiovascular disease, central nervous system disorders including all types of neuropathy, Alzheimer's disease, Parkinson's Disease, diabetes, retinopathy, psychological disorders, kidney disease, the treating of wounds from injury or surgery, and chronic fatigue syndrome.

First among these diseases which respond to the invention as demonstrated in humans, are metabolic disorders and diseases which are not merely diseases of improper blood glucose, but rather diseases of improper body-wide metabolism, both active and resting. By using the invention and oscillations of free hormone (insulin) levels meeting the approximate requirements for the above calories of glucose, the resting metabolism of every person, including a diabetic person can be beneficially shifted from elevated lipid and free fatty acid based, to greater carbohydrate based and normal or even greater than normal adenosine triphosphate levels, among other results. The use of the invention for metabolic diseases addresses the core problem which causes disruption of cellular activities and the multitude of so-called “poor outcomes” associated with improper metabolism, diabetes, Alzheimer's disease, Parkinson's disease and general poor cellular energy levels.

In order to demonstrate that increased adenosine triphosphate production could be achieved by the invention, the Bionica PCA 110 infusion device was modified to provide oscillations with changes in the duration and amount of hormone infused. The resulting device was subsequently used in connection with a range of oral carbohydrates approximating that which is normal for the weight and body mass of the patient. Testing led to developing a desirable infusion and duration adjustment matrix for patients of various weights and hormone sensitivities. Achieving the desired increased adenosine triphosphate levels and metabolic changes using this method was produced by experimentation to identify useful oscillations necessary to induce the metabolic response.

The current invention avoids slip-stick, chatter, overruns and the problem of hysteresis by breaking the seating forces in a lateral motion. This allows for extraordinary accuracy while using common plastics in the pump and control mechanism. Accuracy of plus or minus one percent of one microliter over a ten milliliter range is not uncommon with the preferred device. The preferred device achieves this without error correcting software or other volumetric measurement and control systems. This avoids components which might damage the hormones being infused.

This invention does achieve the varying hormone rates found in a more natural course of physiologic responses to carbohydrates, the fuel which provides a greater amount of adenosine triphosphate than any other fuel. Conventional hormone delivery systems of giving glucose to cover insulin subcutaneously are ineffective in providing normalized adenosine triphosphate. This invention thus provides a basic and significant change from prior therapies where infusions were given in set amounts, and counter-reagents adjusted to keep the patient from excursions outside of customary levels.

In one embodiment of the current invention, the device reverses infusion to withdraw blood samples and allow for the measurement of substrates from whole blood on a real time basis. This avoids manual monitoring of blood conditions and increases the speed and safety of achieving the desired therapeutic changes.

Measurements controlling the infusion timing and amounts can be either by direct measurement or indirectly by the half-life or absorption of the hormone. But to be effective, the infusion preferably achieves oscillations of hormones, which can be measured through whole blood analysis.

Very accurate infusion delivery is beneficial for the invention to overcome the diluting effect of the normal movement of blood in veins. In order to deliver the treatment in discrete boluses, and achieve oscillations of the type needed as shown by this invention, a very accurate intravenous infusion preferably is given. Volume errors can cause a reduction of the range of oscillations, as well as results in changes to the gradient slope of each delivery.

The current invention is an improvement over prior art systems with complicated pumps. The current invention has only one moving part in relation to the delivery mechanisms. Simplicity allows for more accuracy and lower costs. It also allows for a single handed adoption of the Cassette to the Pumping Device, freeing the operator's other hand and avoiding accidental sticking with “sharps” such as needles which are contaminated with blood or other materials.

The preferred embodiment devices avoid slip-stick, chatter and the forces of hysteresis, and still have no gates or valves to damage proteins. Further, it is designed to also be used in a bi-directional application. One of the preferred embodiments herein is the reversal of the Plunger. This can allow for a precise amount of blood to be withdrawn, and be made available in the closed system, particularly to be tested. The preferred embodiment uses this bi-directional control to allow the device to be fitted with a variety of probes in line. This can provide access to measure the properties of a sample, such as blood. The sampled volume can be re-infused back to the zero point, or if desired, sent through a second flow direction. This second direction may including depositing that tested blood into a separate container or depository or directed to waste.

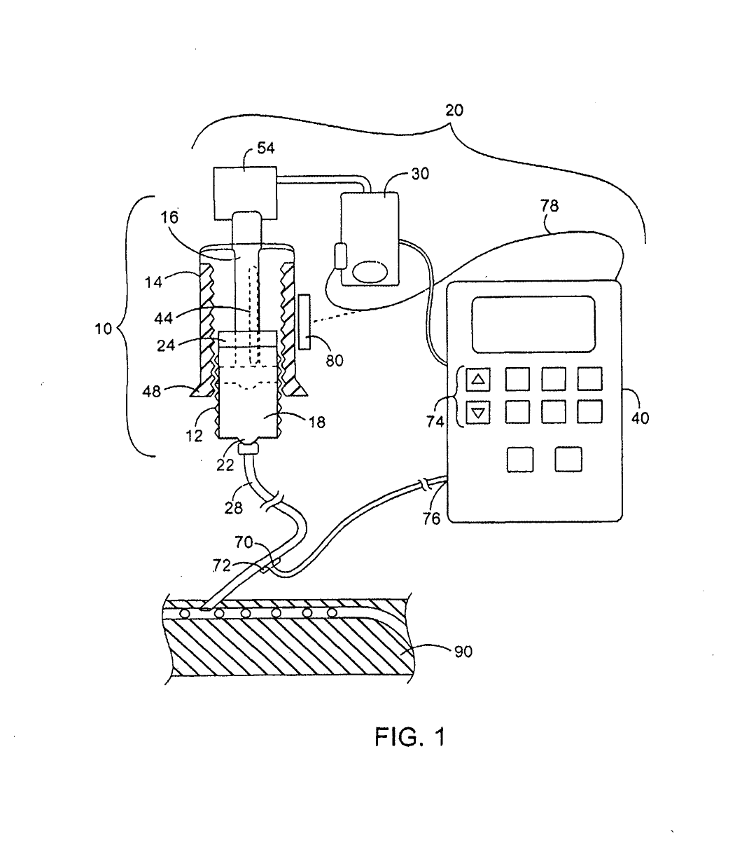

The pumping mechanism preferably is actuated by a mechanical system which rotably moves the plunger or housing, depending upon the configuration. FIGS. 1, 2 and 3 show a number of different ways to provide this two-axis motion in relationship to the Plunger and Housing. The current invention allows for direct drive, stepper motor, or even spring motor to deliver the amount of movement desired. The “motor” (driving force) can be even a coordinated hand-eye movement. Movement can be to a series of “click” points.

The reservoir in the preferred embodiment can be pre-filled. This, for example, can enable the seller to standardize pre-filled reagent cartridges. Also, expensive residues of unused hormones (left-overs) are not necessarily discarded. And there is no need for filling from a container or handling of an additional bottle as pre-filling allows for no filling or common source waste. The cassette can be removed and re-inserted to store the unused reagent for an appropriate period of time in the Cassette.

The design of the motor and assembly allows the pump to be put above, at, or below the heart level, with no resulting change in the delivery profile. This allows the pump to be worn or enclosed in several different tamper-proof or patient access limiting configurations.

The preferred embodiment cartridge, when engaged in the delivery device, locks by the rotational providing threads and this locking of the meshed threads makes an accidental infusion by dropping or pressing on the plunger virtually impossible. The cartridge resists siphoning out of the pump, or accidentally delivering fluid when dropped or pushed against.

Since the cartridge can also be the pumping system, each time such a cartridge is used, it can be replaced, and the entire wearing aspects of the pumping system are replaced, thereby permitting the product life cycles to be much greater. The entire fluid handling system preferably is replaced with each use, thus eliminating the need for sterilizing and cleaning of parts.

All of the foregoing attributes of the invention and preferred embodiment are beneficial to inducing improved adenosine triphosphate production, and the providing of the preferred embodiment includes all of the above attributes.

Accordingly, the present invention is a medical infusion system which may or may not have an aspiration system, which infusion system delivers hormone infusions of accurate bolus deliveries at relatively high rates of flow which approximate hormone secretion rate changes necessary and sufficient to cover the action of glucose or other carbohydrates in an amount equal to over 40% of the amount of carbohydrates the patient would take as suggested by standard daily values for the weight and body mass of the patient. There is not a specific range of carbohydrate loading. Approximately 20% to approximately 60% should be useful, and perhaps even wider ranges.

The delivery profile is designed to allow increasing or decreasing free levels of hormones by infusion in boluses in accordance with the clearing of the hormone by the patient's body.

The purpose of the preferred embodiment is to provide a system of individual boluses of differently spaced and with different amounts of infusion, to respond to changes in the body, such as the amount of free glucose in whole blood.

The preferred embodiment avoids the mechanical slip-stick, chatter, overruns, and the problem of hysteresis by breaking the seating forces between plunger and cassette wall in a lateral motion that does not vary the delivery profile, and overcomes viscosity forces of the reagent. The system also eliminates the need to dilute to provide additional control for the limitations of accuracy in other systems. Other important characteristics of the preferred embodiment include disposability, inexpensive cost and use by the manufacturer in glass lined or plastic devices, and for the cassette to act as both the pumping cartridge and the shipping and storage cartridge thus avoiding loss of reagent in the priming of an infusion device. The preferred embodiment also eliminates the need for withdrawing the medicine with a needle and achieves extraordinary accuracy without error correcting software or expensive volumetric measurement and control systems.

The present invention is a medical infusion and possible aspiration system capable of accurate bolus delivery at relatively high rates of flow adjusting the time between boluses and amount of each bolus to provide the required infusion stimulation and resulting oscillations. The present invention provides a means to provide changes in timing and amounts of delivery to provide maximum tissue stimulation while automatically avoiding errors in concentration, reagent and hormone type, and avoiding the problems of shear and other hormone degrading pressure problems. The system can also avoid the slip-stick, chatter, overruns, and the problem of hysteresis by breaking the seating forces between the plunger and cartridge wall in a lateral motion that does not vary the delivery profile by the viscosity of the reagent. The system can also avoid any loss of reagent which occurs in the priming of a cartridge and eliminate the tendency of reagents to separate when in a diluted environment. The invention is disposable, can be inexpensive and may be used by the manufacturer in glass lined or plastic, as both the pumping cartridge and the shipping and storage cartridge. In general, the current invention comprises an infusion pump and possible cassette cartridge pumping and aspirating device. The cassette cartridge pumping and aspiration system consists of a cartridge, a housing, a plunger, a reservoir area where the reagent is contained, a neck opening in the plunger or cassette for the connection of the cartridge to a tube which travels to where the infusion takes place, and an in-line area where probes for sampling may be located. The in-line area probes can be used to provide input to a pumping device. The preferred embodiment has only one moving part in relation to the delivery mechanism and this simplicity allows for more accuracy and lower costs. It also allows for a single handed adaptation of the cartridge to the pumping device, freeing the other hand and avoiding accidental sticking with “sharps” such as needles which may be contaminated with blood or other materials.

The current invention consists of a pump, and in the preferred embodiment a cassette cartridge pumping and aspirating system. The cassette cartridge contains a plunger, a reservoir area where the reagent is filled, a neck opening for the connection of the plunger or cartridge to a tube which travels to where the infusion takes place, and an in-line sensor area where probes for sampling are located. The in-line area probes can be used to provide input to the pumping device. The preferred embodiment has only one moving part in relation to the delivery mechanism. Simplicity allows for more accuracy and lower costs. It also allows for a single handed adaptation of the cassette to the pumping device, freeing the other hand and avoiding accidental sticking with “sharps” such as needles which are contaminated with blood or other materials.

In a preferred embodiment, the cartridge is cylindrical in shape and has an encoded area. The cartridge may be made of glass, plastic steel or ceramics. It is preferable that part of the outer surface of the cartridge be threaded, and part of the outer surface be grooved to accept and mate with a turning gear. The reservoir area is preferably used for containing a reagent and may be pre-filled.

The neck opening, when not contained in the plunger, is preferably located at the bottom surface of the cassette cartridge and sized to connect an infusion tube to the cartridge. Any conventional tube connection device may be used to connect the infusion tube to the cartridge. The cartridge also has a cap and container top which protects the plunger and its opening, and allows the cartridge to act as the storage vessel for the reagent, and thereby avoid additional steps of filling, mixing, measuring or wasting reagent in the handling of the fluid.

In the preferred embodiment, an optical or electromagnetic strip is located within an encoded area on the cartridge. When the cartridge is filled, an optical or electromagnetic strip with information on the contents and uses of the reagent is placed in the encoded area. The encoded area is preferably located on the outer surface of the cartridge in the area that is first inserted the housing. When the cartridge is placed in the device, it is preferable that the rotational action causes the encoded area to be well aligned and easily read with the uniform motion of screwing the cartridge into place. The preferred rotation, pre-determined position of the encoded area, and the ease of programming a unique character to each cartridge allows the reagent to be mistake limiting.

The preferred embodiment system benefits from entering the weight of the patient into the pumping device control system, thus reducing the incidence of errors. Any conventional method of storing and retrieving data from the encoded area are preferably included in the present system to limit the incidence of errors. It is preferable that the encoded area comes into close proximity with a reading system as the cartridge is loaded or is first used. The reading system may be any commercially available system capable of reading the encoded area. A medical device stores and uses the encoded information in its operations, including a means to limit the profile of the infusion allowed without further intentional override of the profile.

In the preferred embodiment, the housing consists of a cylindrical tube that is sealed at the upper end, made from plastic and opens at the bottom end to allow cartridge removal. The inner surface of the housing is preferably threaded and sized to receive the cartridge. A plunger is preferably connected to the sealed end and is suspended in alignment with the central axis of the cylindrical tube by a stanchion. In the preferred embodiment, there is a plurality of openings cut through the housing to allow for normally trapped air to be exhausted as the plunger either advances or retards. The plurality of openings also creates an inspection window within the housing allowing visual access and access to the optical or electromagnetic strip within the encoded area. A lip at the bottom of the housing provides for a manually removable cover used to protect the cartridge from contamination or damage to the plunger. When the cartridge is engaged in the housing, the cartridge is locked into place by the rotational engagement of the threads. The locking of the meshed threads makes an accidental infusion by dropping or pressing on the plunger virtually impossible. The cartridge will not siphon out of the pump, or accidentally deliver fluid when dropped or pushed against, which overcomes a major disadvantage to conventional cartridges or syringes.

The preferred plunger is a piston-type plunger and is preferably connected by a stanchion to the sealed end of the housing and is aligned with the central axis of the housing. The plunger head is preferable concaved to trap air and allow for bubble removal, and when an opening is provided on the cartridge, the plunger is sized to fit any reservoir opening, so there is very little dead space thus resulting in very little loss of reagent in the final stroke or at the end of treatment.

In the preferred embodiment, a mechanism is used to rotate the plunger within the cassette and thereby infuse the appropriate amount of hormone to achieve increased adenosine triphosphate and related metabolic actions. The mechanism comprises a gear linkage to the motor, a motor and a gear connection to the cartridge which acts as the pumping device. The pumping mechanism may be actuated by any motor which rotably moves either the plunger or housing, with the other of the two fixed. The present invention allows for direct drive, stepper motor, spring or band action motor, or hand articulation to deliver the desired plunger rotation. The “motor” may be even a coordinated hand-eye movement or movement to a series of “click” points. In a preferred embodiment, the plunger rotates in relation to the walls of the cartridge.

In the preferred embodiment, the cartridge, when placed in the housing, causes the piston plunger to move both forward and aft to infuse or aspirate, and at the same time rotate the plunger within the cartridge to break the forces of inertia and slip-stick as well as eliminate backlash. Because the device avoids slip-stick, chatter and the forces of hysteresis, and has no gates or valves, it is designed to also be used in a bi-directional application, such as one of the preferred embodiments herein, where the precise amount being withdrawn may be distributed, equally or in successive steps of precise delivery, or the precise amount withdrawn re-inserted into the patient to the “zero” point.

A sensor area located in the infusion tube contains probes designed to determine the chemical components and levels of desired substrates in the aspirated whole blood. The determination can be by direct measurement of the medicine, or by measurement of the response of the patient to the medicine. The information obtained by the probes relayed to the pumping device and is used to control or limit the infusion profile, including the changing of both the time between pulses and the amount pulsed.

The bi-directional accuracy of the present invention allows the system to withdraw a measured sample and be used with any number of probes. It is preferable that the probes measure the properties of a sample, such as blood, and then allow the present invention to re-infuse that sample back into the patient after it has been tested which is safe since the system is closed to outside contaminants, or if necessary, by second valve or gate, deposit that blood into a separate container or depository.

The present invention also includes a means to control the cartridge. The pumping system preferably has one, two, three or more sources of input. The preferred pumping device includes, but is not limited to, an input system to drive the device, a sensor input for in-line measurement of substrates, an in-line occlusion pressure sensing system and/or input from the reading of the encoded area, and a means for providing increased adenosine triphosphate. The sensor input receives signals from the in-line sensor probes. The in-line occlusion pressure sensing system determines the line pressure or back pressure on the motor. Other traditional pump features are intended to be incorporated into the pumping device. In the preferred embodiment, the rotational velocity exceeds the axial velocity, although with sufficient diameter the difference in rotational travel to axial travel could be adjusted for the flow characteristics of the fluid to be infused and aspirated.

Since the cartridge also acts as the pump, each time the cartridge is used, it and preferably the plunger are replaced, and the entire wearing aspects of the pumping system are replaced. The product life cycles are much greater, since the entire fluid handling system is replaced with each use, and sterilization and cleaning of parts is eliminated.

The purpose of the present invention is to provide improved adenosine triphosphate production by providing measured and spaced boluses of hormones, including insulin, which result in oscillations of free levels of hormones in blood approximating the amount needed to cover at least 40% of a standard carbohydrate meal for that patient. The need to have stimulation like the dynamic homeostasis which produces adenosine triphosphate from carbohydrates was deemed by the Inventor to be a valid approach to induce increases in available cellular energy and thus help the cells to achieve the above therapeutic outcomes.

The pumping and aspirating device as seen in FIG. 1, is an embodiment of the invention delivering the desired hormone pulses resulting in oscillations which provide the necessary dynamic relationship between rising glucose and oscillations of hormones in the whole blood of the patient. The cassette device with a plunger, a cylinder area where the reagent is filled, a neck opening in the plunger for the connection of the cartridge to a tube which travels to where the infusion takes place, and the in-line area where probes for sampling can be located to provide input to the pumping device, are additional aspects of the invention which help to provide improvements over the basic unique delivery modality.

The Housing can either turn or be affixed to the “Pumping Device” with gearing to link the plunger, cassette, or housing to the motor. The motor can be either electromechanical or a manual wind up, spring or band action motor, adjusted by a mechanical timer for the delivery profile. The motor can also be the manual turning by a hand.

The pumping device can have one, two, three or more sources of input, beginning with the input system to drive the device (“Input”) and a possible input of sensors for in-line for measurement of substrates (“In-line Sensor Probes”) an in-line occlusion pressure sensing system from the line pressure, or an occlusion sensor from back pressure on the motor, (“Occlusion”) and/or input from the reading of the encoded area. Other traditional pump features are intended to be incorporated into the final pumping device system.

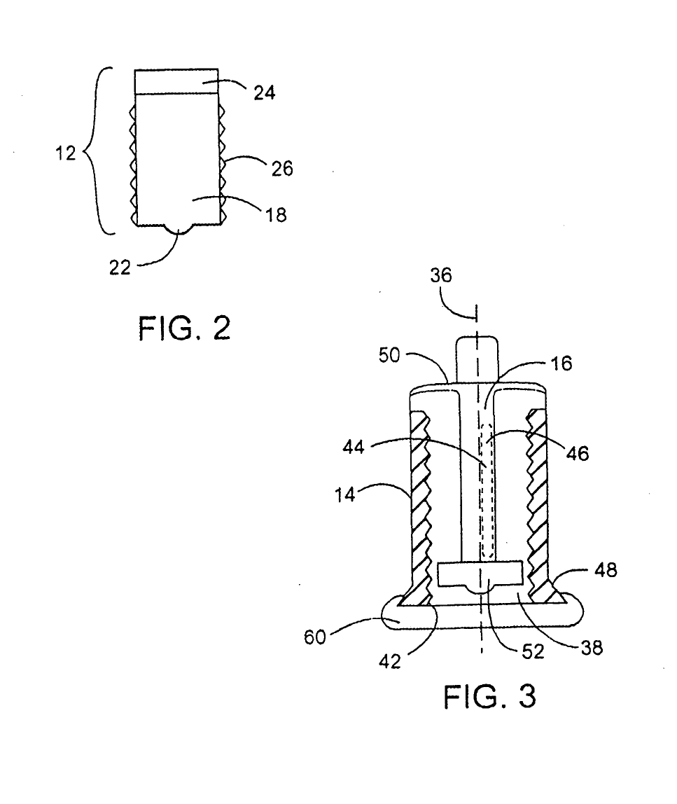

FIG. 2 shows the Cassette with only one opening, where the plunger also provides the locking connection system to the infusion tube. A standard luer lock attachment, or other quick connection system would be included in the plunger as a single piece. A “Protective Cap” is shown removed from the single piece cassette. Splines or grooves on the side of the cassette mesh with a gearing mechanism driven by the Motor, all of which are attached to the “Pumping Device” system. The motor rotates the cassette by attachment to splines on the side, with the housing fixed to the pumping device. The plunger then rotates in reference to the cassette, due to the locking of the plunger to the housing by the stanchion.

FIG. 3 shows a direct screwing interface to the side of the cassette to accomplish the rotational and axial movement required to provide the delivery profile, as well as a clamshell opening system for easy removal of the cassette and an internal stanchion to hold the plunger which automatically causes the plunger to turn in reference to the cassette as the motor advances the cassette upwards and downwards in order to infuse and aspirate.

In the preferred embodiment, the rotational velocity exceeds the axial velocity, although with sufficient diameter the difference in rotation travel to axial travel could be adjusted for the flow characteristics of the fluid to be infused and aspirated.

Housings except as to one version of the rack as shown in FIG. 3, can be made in a clamshell or disassembled manner for easy withdrawal.

Referring now to the figures, FIG. 1 is a perspective view of an embodiment of the invention showing a cassette 10, a pumping mechanism 20 and a motor 30. The cassette further comprises a cartridge 12, and a housing 14.

In a preferred embodiment, as best seen in FIGS. 1 and 2, the cartridge 12 is cylindrical in shape and has a reservoir area 18, and encoded area 24. The cartridge 12 is preferably made from glass or plastic. For high-pressure situations, it is preferable that the cartridge 12 be made of steel or ceramics. It is preferable that the outer surface of the cartridge be partially threaded at the top and grooved for the remaining area to the end of the cartridge 26. Any standard or metric thread and groove sizes may be used.

The reservoir area 18 is preferably used for containing a reagent. The reservoir area 18 may be pre-filled, thereby enabling the seller to market pre-filled reagent cartridges which also act as the storage and transportation vehicle. The preferred embodiment eliminates expensive residue that is thrown away with a separate transportation bottle, as pre-filling allows for no waste. The preferred embodiment eliminates dilution requirements due to the accuracy of the pumping means. Cartridges which may be re-inserted can store the unused reagent for an appropriate period of time in the cartridge.

The housing is preferably opened along its center axis to remove cartridges, by means of a hinge 51 which is located off of center to allow the tube to run unobstructed up the stanchion and out of the cassette.

The neck opening 22 is preferably located in the plunger 52, or may be located on the bottom surface of the cartridge 12. The neck is preferably sized to connect an infusion tube 28 to the cartridge 12. Any conventional tube connection device may be used to connect the infusion tube 28 to the cartridge 12. The opposite end of the infusion tube 28 is connected to the sensors and then to a vein in the patient 90.

It is preferable that the cartridge 12 also contains a cap as the container top which allows the cartridge 12 to act as the storage vessel for the reagent, and thereby avoids the additional steps of filling, mixing, diluting, measuring or wasting reagent in the handling of the fluid.

In the preferred embodiment of the invention, an optical or electromagnetic strip is located within an encoded area 24 on the cartridge 12. When the cartridge 12 is filled, an optical or electromagnetic strip with information on the contents and uses of the reagent is placed in the encoded area 24. The encoded area 24 is preferably located on the outer surface of the cartridge 12 in the area that is first inserted the housing 14.

It is preferable that optical reading of a bar code or other reading of the encoded area 24 will minimize dosage mistakes, as each cartridge can set the maximum allowable dose or delivery. When the cartridge: 12 is placed in the system, it is preferable that the rotational action causes the encoded area 24 to be well aligned and easily read with the uniform motion of screwing the cartridge 12 into place. The preferred rotation, pre-determined position of the encoded area 24, and the ease of programming a unique character to each cartridge 12 allows the reagent to be mistake limiting. Furthermore, the preferred embodiment system requires a weight to be entered into a pumping device 40 for each patient, and when computed with the allowable dosing based on weight, greatly reduces the incidence of errors. Any conventional method of storing and retrieving data from the encoded area are preferably included in the present system to limit the incidence of errors.

It is preferable that the encoded area 24 comes into close proximity with a reading system as the cartridge 12 is loaded or is first used. The reading system may be any commercially available system capable of reading the encoded area 24. A medical device stores and uses the encoded information in its operations, including a means to limit the profile of the infusion allowed without further intentional override of the profile.

In the preferred embodiment, the housing 14 consists of a cylindrical tube that is sealed at the upper end, as shown in FIG. 3. The housing 14 is preferably made of plastic, however, any suitable commercially available material may be used. The bottom 38 of the housing is preferably open and the inner surface 42 of the housing is threaded. Any standard or metric thread size may be used. A plunger stanchion 16 is preferably connected to the sealed end 50 and is suspended in alignment with the central axis 36 of housing 14. The plunger stanchion is fixed to the housing and is mated with the plunger 52 when the cartridge 12 is inserted into the housing 14. The plunger 52 is fixed to the stanchion and is not allowed to rotate with the cartridge is turned. The housing 14 is sized to threadedly receive the cartridge 12. In the preferred embodiment, there is a plurality of openings 44 cut through the housing 14 parallel to the central axis 36 of housing 14. These openings 44 allow for normally trapped air to be exhausted as the plunger 16 either advances or retards. The plurality of openings 44 also creates an inspection window 46 within the housing 14. The inspection window 46 also allows access to the optical or electromagnetic strip within the encoded area 24. A lip 48 at the bottom 38 of the housing 14 provides for a manually removable protective cap like that used for the cartridge 60 used to protect the housing and the cartridge from contamination or damage to the plunger 16.

When the cartridge 12 the plunger 52 engages the stanchion 16 to lock it into place and then when the cartridge is then engaged in the housing 14, the cartridge 12 is locked into place by the rotational engagement of the threads 26, 42. The locking of the meshed threads makes an accidental infusion by dropping or pressing on the plunger virtually impossible. The cartridge 12 will not siphon out of the pump, or accidentally deliver fluid when dropped or pushed against.

The preferred plunger 52 is a piston-type plunger and is made from plastic, however, any type of non-reactive material may be used. The plunger 52 is preferably connected to the sealed end 50 of the housing 14 and is aligned with the central axis of the housing. The plunger 52 preferably has a concaved face to allow any air to first fill the neck space and be eliminated when the cartridge 12, is inserted into the housing 14, and is preferable sized to fit within the reservoir area 18, so there is very little dead space thus resulting in very little loss of reagent in the final stroke or at the end of treatment.

The plunger 52 and reservoir area 18 configuration may have a larger diameter in relationship to the depth the plunger travels, or a very small diameter and longer plunger travel, depending upon the flow characteristics desired for the application. In very viscous fluids, a different diameter would be helpful for both storage and delivery reasons.

In the preferred embodiment, a pumping mechanism 20 is used to rotate the cartridge with the stanchion 16 and the plunger 52, fixed to the housing 14. Grooves 62 on the side of the cartridge 12 mesh with the gearing mechanism driven by the motor 30, all of which are attached to the pumping device 40. The motor 30 rotates the cartridge 12 by attachment to the grooves 62 on the side of the cartridge, with the housing 14 fixed in relation to the motor or pumping device.

The pumping mechanism 20 comprises a gear linkage 54, a motor 30 and a pumping device 40. The pumping mechanism 20 may be actuated by any motor which rotably moves the stanchion 16 and plunger 52, or rotates the housing 14. The present invention allows for direct drive, stepper motor, spring or band action motor, or hand articulation to deliver the desired plunger rotation. The “motor” may also be a coordinated hand-eye movement or movement to a series of “click” points. In a preferred embodiment, the stanchion 16 and plunger 52 rotate in relation to the walls of the cassette 12.

In one embodiment, a motor 30 with either electromechanical or mechanical operation causes a rotation of the cartridge 12 with the stanchion 16 and plunger 52 fixed to the housing 14, giving both lateral and axial movement of the stanchion 16 and plunger 52. The motor 30 is controlled by an input to cause the pumping and aspiration actions to take place as desired to achieve the free medicine profile. In the case of a mechanical motor, the settings may be made by a spring-like mechanism, with the number of turns and speed of the mechanism being governed by a simple clock mechanism.

The design of the motor 30 and assembly allow the pump mechanism 20 to be put above, at, or below the heart level, with no resulting change in the delivery profile. This allows the pump mechanism 20 to be worn or enclosed in several different tamper-proof or patient access limiting configurations.

The planes formed by the inner surface 42 of the housing and part of the outer surface 26 of the cartridge are positioned so as to allow the cartridge 12 to begin turning as it is first attached, or after the plunger 52 is attached to the stanchion 16. The stanchion 16 may extend beyond the line of the housing 14 for purposes of easy snap-in connection and alignment of the plunger 52 and also the cartridge 12. The number of turns per meter or inch are adjusted to provide the desired rate of flow in both directions. The diameter of the cartridge and its separate housing are adjusted to provide different flow rates and to adjust for any necessary fluid dynamics which might be necessary to pump highly viscous liquids or pump fluids at high flow rates.

As the cartridge 12 is rotationally turned, the device infuses or aspirates liquid, depending on the rotational direction of rotation. The rotational movement of the present invention allows for bi-directional movement and provides accurate infusion or aspiration.

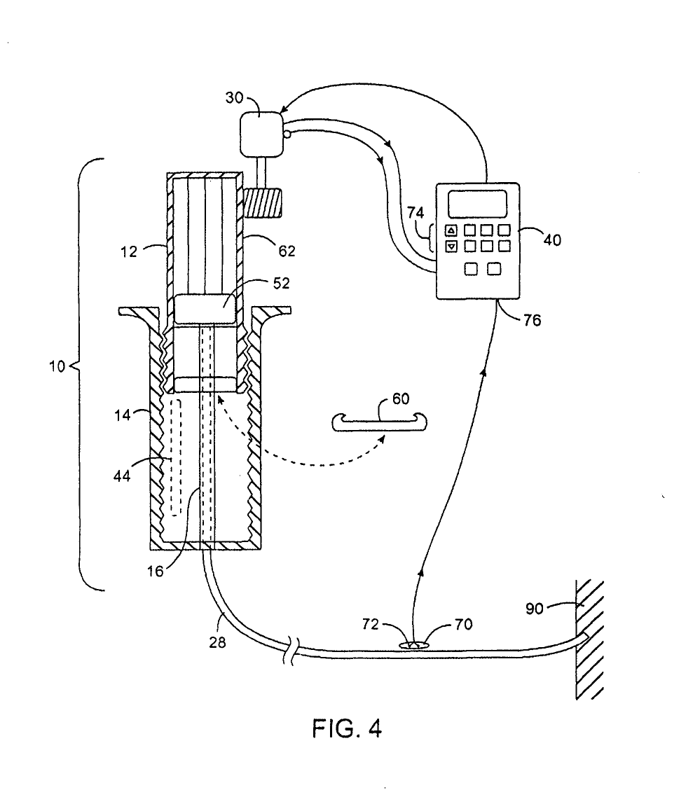

FIG. 4 shows an alternate embodiment of the device. In this embodiment, the cartridge 12 has an opening 22, where the infusion tube 28 is connected to the cartridge. The plunger 16 then rotates, due to the lands and groves meshed between the housing 14 and cartridge 12, causing the infusion and aspiration.

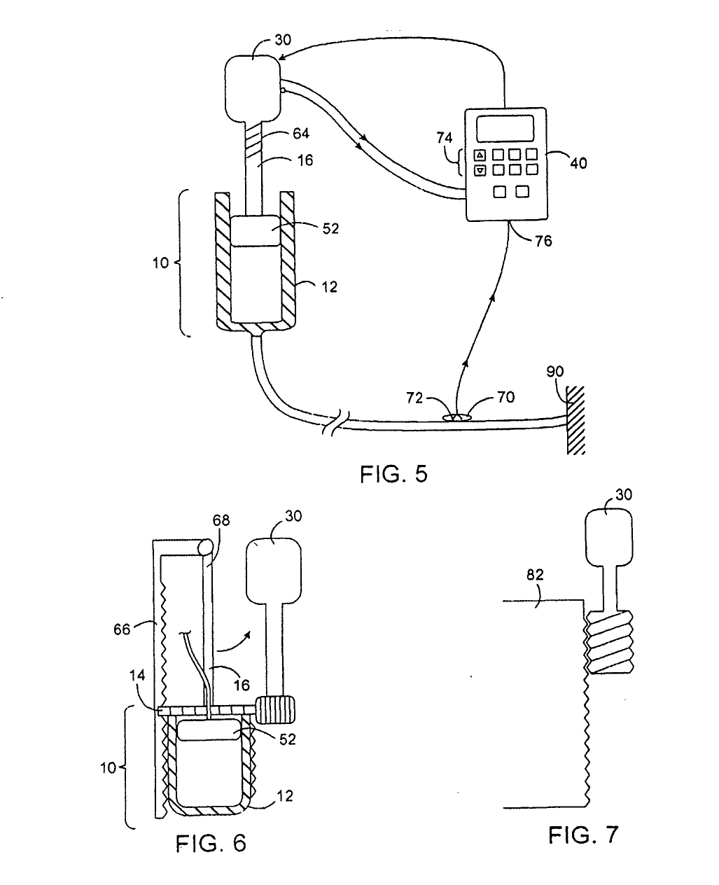

FIG. 5 shows another embodiment of the present invention. In this embodiment, a direct screwing system 64 interface is attached to the side of the cassette 10. The direct screwing system 64 accomplishes the rotational and axial movement of the plunger 52 by dual gearing of the internal rotational drive which automatically causes the plunger 16 to turn as the motor advances the plunger 16 downwards and upwards in order to infuse or aspirate.

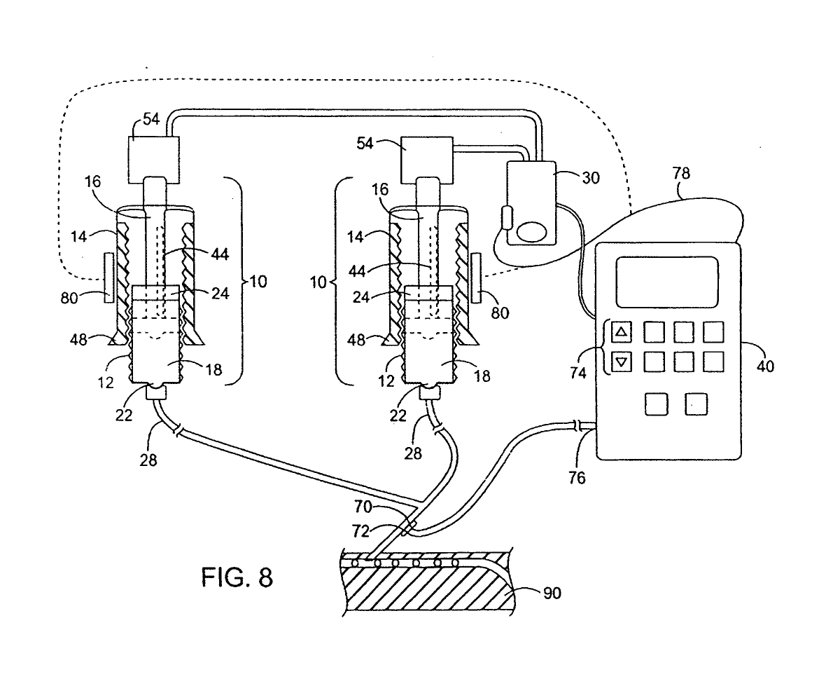

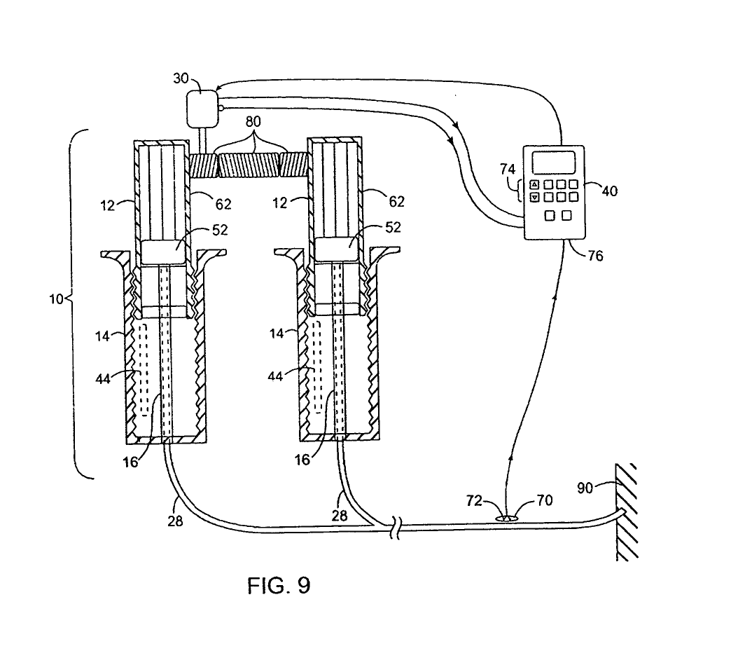

FIG. 6 shows a fourth embodiment of the present invention. FIG. 6 shows a rack 66 threaded surface, which allows the motor 30, when placed adjacent to the rack 66, to turn the housing 14. The plunger 16 remains stationary in relation to the motor 30 and rack 66, thereby causing the plunger 16 to move rotationally in reference to the cassette 10. The plunger sanction 68 may swing away for easy snap-in and snap-out action. FIG. 7 shows a fifth preferred embodiment of the present invention. FIG. 7 shows a side screw 82 configuration for the cassette. FIG. 8 and FIG. 9 show multiple cassettes.

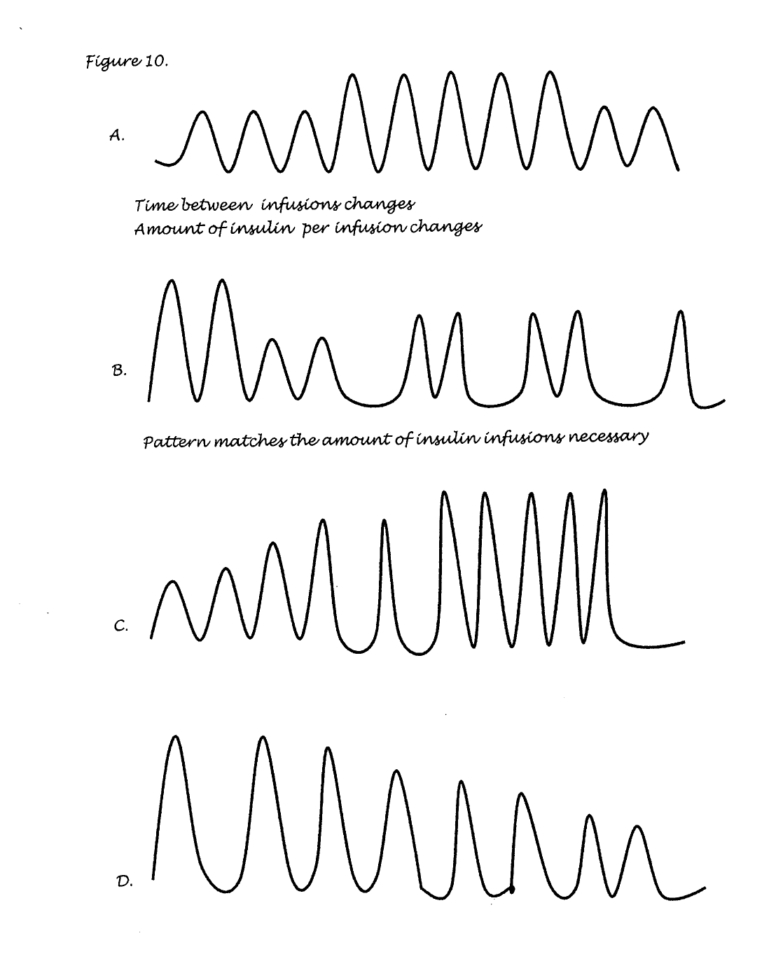

FIG. 10 shows several representative infusion profiles A-D, each where the infusion begins with no or little background level of bioavailable medicine and the time between infusions X is maintained or changed Y with the concurrent amount of each pulse or bolus changed Z. The profile can be changed to provide increased or decreased baselines of free levels of medicines.

In the preferred embodiment, the cartridge 12, when placed in the housing 14, causes the piston plunger 16 to move both forward and aft to aspirate for testing and then infuse, as well as rotate within the cassette 10 to break the forces of inertia and slip-stick as well as eliminate backlash. The infusion is delivered in pulses where the duration between pulses X is changed Y to increase, maintain or decrease the levels of free medicine which causes beneficial responses by tissues to the medicines. Because the device avoids slip-stick, chatter and the forces of hysteresis, and has no gates or valves, it is designed to also be used in a bi-directional application, such as one of the preferred embodiments herein, where the precise amount being withdrawn may be tested and then re-inserted into the patient to the “zero” point.

In the preferred embodiments shown in FIGS. 1, 4, and 5, a sensor area 70 is located within the infusion tube 28. The sensor area 70 contains probes 72 designed to determine the chemical components and levels of desired substrates in the aspirated fluids. The information obtained by the probes 72 relayed to the pumping device 40 and is used to control or limit the infusion profile(s) as contained in FIG. 10. In prototype construction the probes were made of electromagnetic material, however any probe capable of relaying information to the pumping device may be used, including radio frequency, light, infrared waive forms, and chemical testing which is photo sensitive or reactive to the desired substrates.

The bi-directional accuracy of the present invention allows the system to be used with any number of probes. It is preferable that the probes measure the properties of a sample, such as blood, and then allow the present invention to re-infuse that sample back into the patient after it has been tested, or if desired, by second flow direction, deposit that blood into a separate container or depository.

Referring to FIGS. 1, 4 and 5, the present invention also includes a pumping device 40. The pumping device 40 preferably has one, two, three or more sources of input and delivers infusions FIG. 10. The preferred pumping device includes, but is not limited to, an input system to drive the device 74, a sensor input for in-line measurement of substrates 76, an in-line occlusion pressure sensing system 78 and/or input from the reading of the encoded area 80. The sensor input 76 receives signals from the in-line sensor probes 72. The in-line occlusion pressure sensing system 78 determines the line pressure or back pressure on the motor. Other traditional pump features are intended to be incorporated into the pumping device 40.

In the preferred embodiment, the rotational velocity exceeds the axial velocity, although with sufficient diameter the difference in rotational travel to axial travel could be adjusted for the flow characteristics of the fluid to be infused and aspirated.

It is preferable that a second cassette and housing may be coupled and driven either independently or in mechanical linkage 80 with a cassette housing as shown in FIGS. 8 and 9, so as to have as many infusion profiles, either in succession or concurrently as is desired for the given flow profiles and applications, with flows as shown in FIG. 10.

It is a desired effect of the present invention that certain deliveries via long catheters positioned in the patient may benefit from a very stable and accurate system which is not subject to the errors of conventional pumps, even when overcoming higher pressures within a given area.

Since the cartridge and plunger is also the pumping system, each time the cartridge and plunger are used, they are replaced, and the entire wearing aspects of the pumping system are replaced, thereby causing the product life cycles to be much greater. The entire fluid handling system is replaced with each use and sterilization and cleaning of parts is eliminated.

The apparent benefit to having timed boluses of individually controlled amounts, of almost any hormone, as an additional mode for delivery, was deemed by the Inventor to be a valid approach this particular infusion therapy.

### TREATMENT EXAMPLES

1. Male, 55 years old:
One example of a preferred treatment is a 55 year old male weighing 85 kilograms, with decreased resting carbohydrate metabolism as measured by indirect calorimetric measurement, with a decreased volume of Carbon Dioxide Production equaling 0.124 L per 30 seconds of exhaled breath, and a Resting Energy Ratio of 0.66, indicating little or no production of ATP from carbohydrates, and a depress overall resting energy state. After three sessions of one hour treatments (plus rest cycles) by oscillations using the Bionica infusion device and a total of 63 units of insulin in 25 milliunits of insulin per kilo of body weight, the resulting change was an increase in Carbon Dioxide Production to 0.255 L per 30 seconds of exhales breath, and a Resting Energy Ratio of 0.96 indicating approximately 205% increase in ATP from carbohydrates.

2. Female, 74 years old:
Another example of the treatment includes a 74 year old female weighing 70 kilograms with decreased resting carbohydrate metabolism as measured by indirect calorimetric measurement, with a decreased volume of Carbon Dioxide Production equaling 0.091 L per 30 seconds of exhaled breath, and a Resting Energy Ratio of 0.63, indicating little or no production of ATP from carbohydrates, and a depress overall resting energy state. After three sessions of one hour treatment by oscillations using the Bionica infusion device and a total of 45 units of insulin in 22 milliunits of insulin per kilo of body weight, the resulting change was an increase in Carbon Dioxide Production to 0.293 L per 30 seconds of exhales breath, and a Resting Energy Ratio of 0.98 indicating approximately 320% increase in ATP from carbohydrates.

3. Female, 16 years old:

Another example of the treatment includes a 16 year old female weighing 50.5 kilograms with decreased resting carbohydrate metabolism as measured by indirect calorimetric measurement, with a decreased volume of Carbon Dioxide Production equaling 0.121 L per 30 seconds of exhaled breath, and a Resting Energy Ratio of 0.67, indicating low production of ATP from carbohydrates, and a depress overall resting energy state. After three sessions of one hour treatment by oscillations using the Bionica infusion device and a total of 42 units of insulin in 28 milliunits of insulin per kilo of body weight, the resulting change was an increase in Carbon Dioxide Production to 0.342 L per 30 seconds of exhales breath, and a Resting Energy Ratio of 0.98 indicating approximately 280% increase in ATP from carbohydrates.

4. Male, 76 years old, with Alzheimer's:

Another example is the treatment of a patient with Alzheimer's Disease, male 76 year old weighing 83 kilograms with resting carbohydrate metabolism as measured by indirect calorimetric measurement, with a decreased volume of Carbon Dioxide Production equaling 0.151 L per 30 seconds of exhaled breath, and a Resting Energy Ratio of 0.79, indicating reduced production of ATP from carbohydrates, and a depress overall resting energy state. After three sessions of one hour treatment by oscillations using the Bionica infusion device and a total of 33 units of insulin in 14 milliunits of insulin per kilo of body weight, the resulting change was an increase in Carbon Dioxide Production to 0.208 L per 30 seconds of exhales breath, and a Resting Energy Ratio of 0.99 indicating approximately 38% increase in ATP from carbohydrates.

5. General:

a. In a preferred implementation, these patients:
i. Were checked for serum blood glucose (BG) about every 30 minutes, and for about 4.5 hours.
ii. Were given glucose averaging approximately 1,200 calories over that time.
iii. Were checked for resting metabolism 4 times, once before each treatment cycle (administration, then rest), and one at the end of treatment.
iv. Carbohydrate measurement was of the resting state, asking patients for approximately 30 minutes to sit still, not tensing up muscles or becoming agitated in any way, with no heavy exercise 24 hours before test, no caffeine for 8 hours or smoking for 6 hours prior to tests, no hyperventilating. Exclusion criteria contraindicating measurement include: Respiratory Rate (RR) over 22 breaths per minute, undue talking, coughing, burping, food in the mouth, COPD, asthma, an active virus (cold or flu) or other breathing abnormalities.

The preferred embodiments described herein are illustrative only, and although the examples given include many specifications, they are intended as illustrative of only a few possible embodiments of the invention. Other embodiments and modifications will, no doubt, occur to those skilled in the art. The examples given should only be interpreted as illustrations of some of the preferred embodiments of the invention, and the full scope of the invention should be determined by the appended claims and their legal equivalents.

What is claimed is:

1. A method of stimulating cellular metabolism, comprising:
adding as needed a carbohydrate input to a patient intended to achieve a selected level of blood glucose,
adding as needed a hormone stimulus to encourage the patient's body to consume glucose and stimulate cellular metabolism,
repeating the hormone stimulus on a schedule sufficient to modify cell and to some extent tissue changes that will persist after the stimulus such that the patient is healthier.

2. The method of claim 1 wherein the hormone stimulus is chosen and modified to produce oscillations in blood glucose levels and stimulate increased intracellular adenosine triphosphate production from carbohydrates.

3. The method of claim 2 to stimulate the patient's body to process about 20% to about 60% of a normal carbohydrate meal for a normal person of the patient's status.

4. The method of claim 2 to stimulate the patient's body to process about 40% of a normal carbohydrate meal

5. The method of claim 2 wherein the hormone stimulus is administered as infusion of a bolus of hormone.

6. The method of claim 5 further comprising giving the patient sufficient carbohydrate input to raise blood glucose levels.

7. The method of claim 5 further comprising administering a plurality of controlled boluses of hormone

8. The method of claim 6 further comprising administering a series of controlled boluses over a period of time.

9. The method of claim 7 further comprising
administering a series of controlled boluses over a period of time,
administering a carbohydrate input to promote elevated blood glucose levels,
choosing controlled boluses to decrease blood glucose levels, then allow those levels to rise, then again decrease blood glucose levels repeatedly.

10. The method of claim 9 further comprising
administering a series of controlled boluses over a period of time,
administering a carbohydrate input which is a significant fraction of a recommended daily carbohydrate intake for a patient of that weight,
considering age, gender, and other normal factors,
choosing controlled boluses to decrease blood glucose levels, then allow those levels to rise, then again decrease blood glucose levels repeatedly.

11. The method of claim 10 wherein
the carbohydrate input is more than about 20% and less than about 60% of a recommended daily carbohydrate intake for the patient.

12. The method of claim 10 wherein
the carbohydrate input is on the order of approximately 40% of a recommended daily carbohydrate intake for the patient.

13. The method of claim 9 further comprising
choosing controlled boluses to decrease blood glucose levels, then allow those levels to rise, then again decrease blood glucose levels repeatedly,
where the high and low levels vary around the mean of these levels by more than about 20% and less than about 70%, that is the high level is about 20 to 70% higher than the mean and the low level is about 20 to 70% lower than the mean.

14. The method of claim 9 further comprising
choosing controlled boluses to decrease blood glucose levels, then allow those levels to rise, then again decrease blood glucose levels repeatedly, where the high and low levels vary around the mean of these levels by about 50%, that is the high level is about 50% higher than the mean and the low level is about 50% lower than the mean.

15. The method of claim 9 further comprising
choosing controlled boluses to decrease blood glucose levels, then allow those levels to rise, then again decrease blood glucose levels repeatedly,
where the high level is about approximately 300 mg/dl and the low level is about approximately 60 mg/dl.

16. The method of claim 9 wherein the hormone is insulin.

17. The method of claim 9 wherein the carbohydrate source is glucose.

18. The method of claim 9 wherein the carbohydrate source is a solution of glucose ingested by drinking.
